(19) United States
(12) Patent Application Publication
(10) Pub. No.: US 2021/0327552 A1
(43) Pub. Date: Oct. 21, 2021

(54) MEDICAL WRISTBAND WITH INCREASED EASE OF MANUFACTURING

(71) Applicant: SATO America, Charlotte, NC (US)

(72) Inventor: Christopher A. Jones, Alsip, IL (US)

(21) Appl. No.: 17/231,570

(22) Filed: Apr. 15, 2021

Related U.S. Application Data
(60) Provisional application No. 63/010,313, filed on Apr. 15, 2020.

(51) Int. Cl. G16H 10/65; G06K 1/12; A44C 5/00; H04W 4/029; G06K 15/02; G09F 3/00

(52) U.S. Cl. CPC G16H 10/65; G06K 1/121; A44C 5/0015; G06K 19/06037; G06K 15/028; G06K 15/021; G09F 3/005; H04W 4/029

(57) ABSTRACT

The present disclosure provides a new and innovative medical wristband that can be thermally printed with patient identification features and that also includes location-tracking features. The provided medical wristband includes a wristband component and a separate closure component. The wristband component may be thermally printed using conventional thermal printers because it is not physically attached to the location tracker that would otherwise make the medical wristband too thick for conventional thermal printers. The wristband component may be printed with patient identification information and/or a scannable code accessing patient identification information. The closure component has a location tracker coupled to it, such as a Bluetooth beacon. In one example, the location tracker is welded to the closure component. Accordingly, a patient may wear a single thermally printed wristband that provides medical professionals with both patient identification features and location tracking features.

### PRIORITY CLAIM

The present application claims priority to and the benefit of U.S. Provisional Application 63/010,313, filed Apr. 15, 2020, the entirety of which is herein incorporated by reference.

### TECHNICAL FIELD

The present application relates generally to medical wristbands. More specifically, the present application provides a new and innovative medical wristband that can be thermally printed with patient identification features and that also includes location-tracking features.

### BACKGROUND

Hospitals or other medical environments typically provide patients with wristbands to identify patient information (e.g., name, date of birth, etc.) with respect to a particular patient. For instance, wristbands may be printed (e.g., thermally or laser printed) with patient information physically printed on the wristband and/or with a code that may be scanned (e.g., QR code, RFID). A medical professional can scan the code using a device to access more detailed patient information that cannot physically fit on the wristband, such as patient medical history or notes about the patient. In some instances, medical professionals may additionally desire to track the whereabouts of the individual patients within the hospital or other medical environment. To do so, patients may be provided with a wristband that includes a location tracker, such as a Bluetooth beacon capable of communicating with Bluetooth sensors positioned within the hospital or other medical environment.

One way of including a location tracker with a wristband is to weld the location tracker to the wristband. For instance, the location tracker may be welded to the clasp portion of the wristband. Current location trackers suitable for a medical wristband, however, such as a Bluetooth beacon, exceed the thickness limits of thermal printers. Typical location-tracking wristbands therefore cannot be thermally printed. Because certain hospitals or medical environments often or exclusively thermally print identification wristbands, patients are accordingly typically provided with both an identification wristband and a location-tracking wristband.

Providing patients with two separate wristbands increases material costs for the hospital or other medical environment. Providing two wristbands also increases the time nurses or other medical professionals must take to create the wristbands, taking them away from other tasks to care for patients. Additionally, wearing two separate wristbands can be inconvenient for patients and/or irritating to patients' skin. Accordingly, there exists a need for a medical wristband that can be thermally printed with both patient identification features and location-tracking features.

### SUMMARY

The present application provides a new and innovative medical wristband that can be thermally printed with patient identification features and that also includes location-tracking features. The provided medical wristband includes a wristband component and a separate closure component. The wristband component may be printed with patient identification information or a scannable code accessing patient identification information. For instance, the wristband component may be thermally printed. The closure component has a location tracker coupled to it. For example, the location tracker may be a Bluetooth beacon or another suitable location tracker. In one example, the location tracker is welded to the closure component. The closure component is configured to secure the wristband component to a patient's wrist. Accordingly, a patient may wear a single thermally printed wristband that provides medical professionals with both patient identification features and location tracking features.

In an example, a medical wristband includes a wristband component and a closure component. The wristband component includes patient identification information. The closure component includes a location tracking beacon and is configured to secure the wristband component to a patient's wrist. The wristband component is separate from the closure component. And the wristband component is thermally printed.

In another example, a method of assembling a medical wristband includes thermally printing a wristband component including patient identification information. A closure component having a location tracking beacon may be obtained. A first end of the wristband component is positioned adjacent to a second end of the wristband component such that the wristband component is positioned around an object. The first end of the wristband component may then be secured to the second end of the wristband component with the closure component.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates an example medical wristband, according to an aspect of the present disclosure.

FIG. 2 illustrates an exploded view of the example closure component of FIG. 1, according to an aspect of the present disclosure.

FIG. 3 illustrates a cross sectional view of the medical wristband of FIG. 1 in assembled form, according to an aspect of the present disclosure.

FIG. 4 illustrates a perspective view of an example closure component, according to an aspect of the present disclosure.

### DETAILED DESCRIPTION

The present application provides a new and innovative medical wristband that can be thermally printed with patient identification features and that also includes location-tracking features. The provided medical wristband includes a wristband component and a separate closure component that may couple the wristband component to an object such as a patient's wrist. The wristband component may include identification information for a patient. In some instances, the identification information may be in the form of a code (e.g., QR code, RFID) that may be scanned. The closure component includes a location tracker (e.g., a Bluetooth beacon) that may be tracked by an external system. For example, a location of the location tracker may be tracked by a radio frequency network system (e.g., Bluetooth). As compared to typical identification wristbands having location tracking that are too thick to be thermally printed, the wristband component of the provided medical wristband can be thermally printed using conventional thermal printers. This is because the wristband component is not physically attached to the location tracker that would otherwise make the medical wristband too thick for conventional thermal printers. Rather, the wristband component may be thermally printed and then coupled to a patient using a separately constructed closure component having a location tracker.

In an example, the wristband component may be thermally printed on-demand when a new patient is admitted to the hospital or other medical environment. Hospitals or medical environments may then have a supply of closure components with coupled location trackers on hand that may be used with the on-demand thermally printed wristband components. This enables a nurse or other medical professional to quickly print a single wristband component for a newly admitted patient that includes identification information (e.g., QR code, RFID) for the patient, grab a closure component that enables location tracking of the patient, and secure the wristband component to the patient's wrist with the closure component. In this way, a patient may wear a single thermally printed wristband that provides medical professionals with both patient identification features and location tracking features. The provided medical wristband therefore reduces the costs for hospitals and other medical environments as compared to printing two wristbands for patients. The provided medical wristband also reduces the amount of time nurses or other medical professionals must take to create wristbands. It also reduces the number of wristbands a patient must wear, which may reduce a patient's skin irritation.

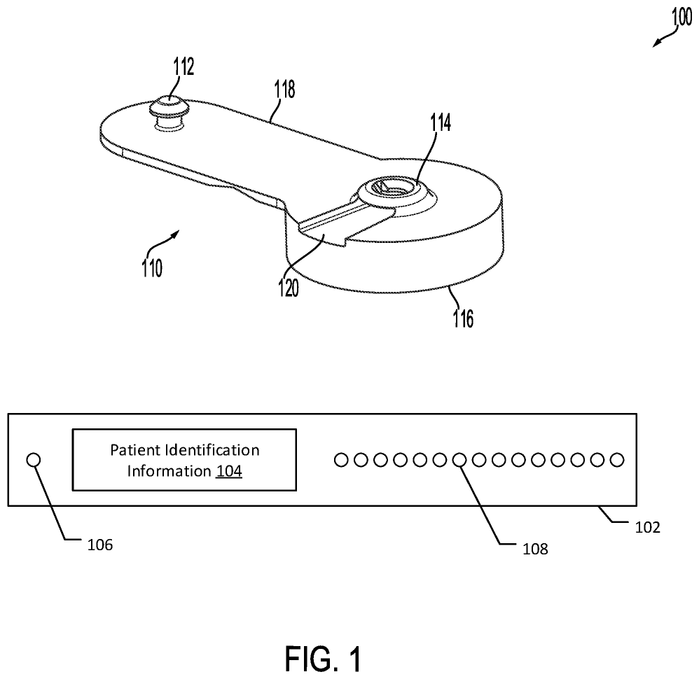

FIG. 1 illustrates an example medical wristband 100. The medical wristband 100 includes a wristband component 102 and a closure component 110. The wristband component 102 is thermally printed. In various aspects, the wristband component 102 is constructed of one or more polymer films, such as polyethylene, polypropylene, polyester, or other poly olefins. The wristband component 102 may be constructed of a single homogenous material or layered composites. The wristband component 102 includes patient identification information 104. For instance, the patient identification information 104 may include identifying information of a patient, such as name, age, date of birth, gender, blood type, and other pertinent information. In some instances, the patient identification information 104 may additionally or alternatively include a code (e.g., QR code, RFID) that a medical professional may be scan with a device (e.g., smart phone, tablet, etc.) to access additional information with respect to a patient. Stated differently, the code may identify electronically stored data that is accessed by scanning the code with the device. For example, a patient's medical history, allergies, symptoms, or a medical professional's notes concerning the patient are typically too lengthy physically include on the wristband component 102. The patient identification information 104 may be printed directly on the wristband component 102, such as when the wristband component 102 is thermally printed.

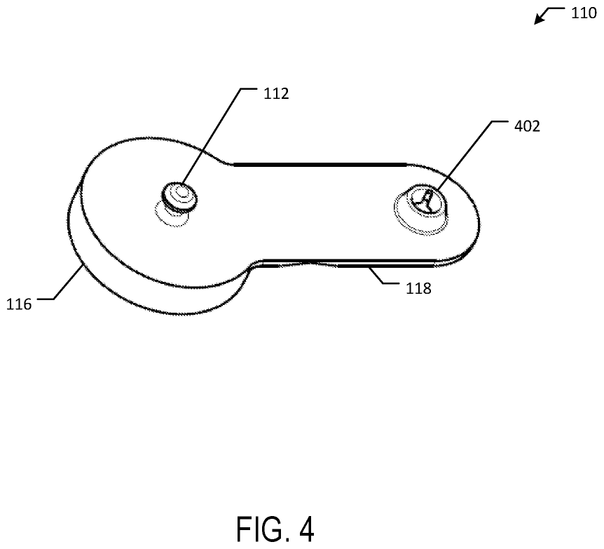

In various aspects, the closure component 110 includes a housing 116. A strap 118 may extend from the housing 116. In some aspects, the housing 116 may include a channel 120. The closure component 110 is configured to secure the wristband component 102 to a patient's wrist. In one example, such as the one illustrated in FIG. 1, the closure component 110 is configured with a snap mechanism. In such an example, the closure component 110 includes a male snap component 112 on one end and a female snap component 114 on its other end. For example, strap 118 may include the male snap component 112 and the housing 116 may include the female snap component 114. In other examples the male snap component 112 and the female snap component 114 may be on opposite ends. For instance, FIG. 4 illustrates an alternative example of the closure component 110 including a housing 116 having a male snap component 112 and a strap 118 having a female snap component 402.

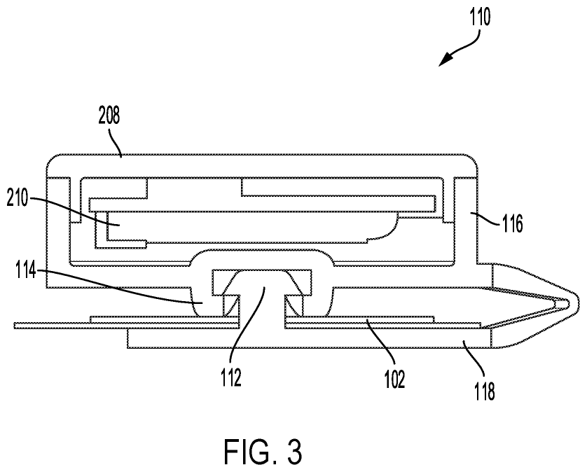

Returning to FIG. 1, in such examples in which the closure component 110 includes a snap mechanism, the wristband component 102 includes a plurality of sizing holes 106 and a securement hole 104. The securement hole 104 may be lined up with one of the sizing holes 106, depending on the size of a patient's wrist, and the male snap component 112 of the closure component 110 may be positioned through the two holes and snap into the female snap component 114 to secure the wristband component 102 to a patient's wrist (e.g., FIG. 3).

In other aspects of the present application, the closure component 110 may be configured in other manners to secure the wristband component 102. For example, the closure component 110 may include two sides that pinch two end portions of the wristband component 102 together so that they cannot move, thereby securing the wristband component 102 to the patient's wrist. In such an example, the wristband component 102 might not include the securement hole 104 and the plurality of sizing holes 106.

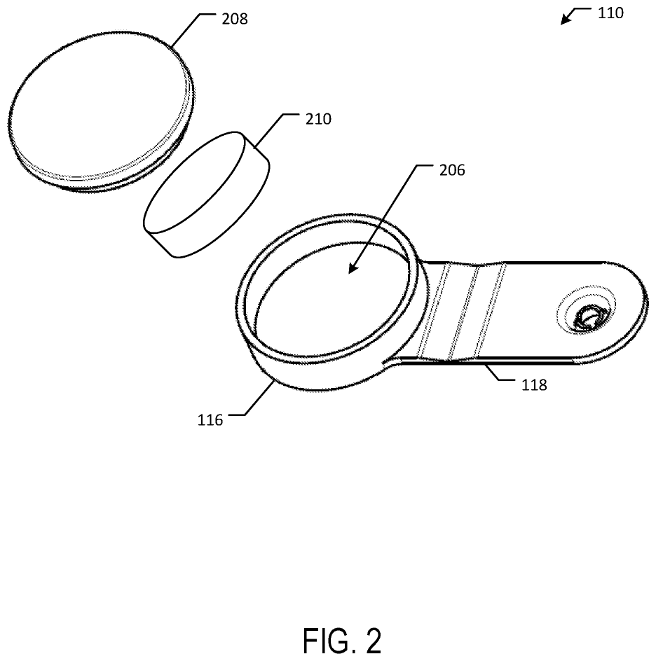

FIG. 2 illustrates an exploded view of the closure component 110. The housing 116 includes an opening 206. A cap 208 may be coupled to the housing 116 to enclose the opening 206. The closure component 110 also includes a location tracker 210. The location tracker 210 is a component of an external location tracking system. For example, a location of the location tracker may be tracked by a radio frequency network system (e.g., Bluetooth). In such an example, the location tracker 210 may be a Bluetooth beacon capable of communicating with sensors. The sensors may monitor where a particular Bluetooth beacon is relative to the sensors within the hospital or other medical environment.

In some instances, the location tracker 210 may also perform other functions. For example, the location tracker 210 enable a medical professional to access detailed patient information that the code of the patient identification information 104 otherwise enabled. In such examples, the patient identification information 104 may solely be physically printed information on the wristband component. In various aspects, the closure component 110 and the location tracker 210 may be constructed of a plastic, such as ABS or other suitable plastics.

The location tracker 210 may be positioned within the opening 206 of the housing 116. In some aspects, the cap 208 enables the location tracker 210 to be removed and reinserted, such as if it ceases functioning and needs to be replaced. In other aspects, the housing 116 may be manufactured around the location tracker 210 (e.g., without a cap 208) such that the location tracker 210 is not removable from the housing 116.

FIG. 3 illustrates a cross sectional view of the medical wristband 100. In this example, the male snap component 112 is shown positioned through the securement hole 104 and the plurality of sizing holes 106 of the wristband component 102 and snapped into the female snap component 114. As such, the wristband component 102 is secured to the closure component 110. Also shown is the location tracker positioned within the opening 206 of the housing 116. The cap 208 encloses the opening 206 in this example.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A medical wristband comprising:
a wristband component including patient identification information; and
a closure component configured to secure the wristband component to a patient's wrist, wherein the closure component includes a location tracking beacon,
wherein the wristband component is separate from the closure component, and
wherein the wristband component is thermally printed.

2. The medical wristband of claim 1, wherein the patient identification information is text printed directly on the wristband component.

3. The medical wristband of claim 2, wherein the patient identification information includes a name, age, date of birth, gender, or blood type of a patient.

4. The medical wristband of claim 1, wherein the patient identification information is a code printed directly on the wristband component, the code identifying electronically stored data that is accessed by scanning the code with an external device.

5. The medical wristband of claim 4, wherein the patient identification information includes a medical history, allergies, or symptoms of a patient, or notes of a medical professional concerning the patient.

6. The medical wristband of claim 1, wherein the wristband component is constructed of one or more polymer films.

7. The medical wristband of claim 6, wherein the wristband component is constructed of polyethylene, polypropylene, or polyester.

8. The medical wristband of claim 1, wherein the wristband component includes a plurality of holes.

9. The medical wristband of claim 1, wherein the closure component includes a housing and a strap extending from the housing.

10. The medical wristband of claim 9, wherein the closure component includes a first snap component located on the housing and a second snap component located on the strap, the first and second snap components configured to snap together.

11. The medical wristband of claim 9, wherein the location tracking beacon is positioned within an opening in the housing.

12. The medical wristband of claim 11, wherein the housing fully encloses the location tracking beacon such that the location tracking beacon is not removable from the housing.

13. The medical wristband of claim 11, wherein the closure component further includes a cap configured to couple to the housing that, when coupled, encloses the location tracking beacon within the opening.

14. The medical wristband of claim 1, wherein the location tracking beacon is a component of a radio frequency network system that tracks a location of the location tracking beacon.

15. The medical wristband of claim 14, wherein the radio frequency network system is Bluetooth.

16. A medical wristband comprising:
a thermally printed wristband component including patient identification information printed on the thermally printed wristband component; and
a closure component configured to secure the wristband component to a patient's wrist, the closure component including:
a housing including a cavity,
a strap extending from the housing, and
a location tracking beacon positioned within the cavity of the housing,
wherein the wristband component is separate from the closure component.

17. The medical wristband of claim 16, wherein the patient identification information is text or a code identifying electronically stored data that is accessed by scanning the code with an external device.

18. The medical wristband of claim 16, wherein the location tracking beacon is removable from the cavity of the housing.

19. A method of assembling a medical wristband comprising:
thermally printing a wristband component including patient identification information;
obtaining a closure component having a location tracking beacon;
positioning a first end of the wristband component adjacent to a second end of the wristband component such that the wristband component is positioned around an object; and
securing the first end of the wristband component to the second end of the wristband component with the closure component.

20. The method of claim 19, wherein the object is a wrist of a patient.
